(19) United States
(12) Patent Application Publication
(10) Pub. No.: US 2020/0055922 A1
(43) Pub. Date: Feb. 20, 2020

(54) IMMUNOGLOBULIN PRODUCTS FOR USE IN THE TREATMENT OF CHRONIC INFLAMMATORY DEMYELINATING POLYNEUROPATHY

(71) Applicant: CSL BEHRING AG, BERN (CH)

(72) Inventors: ORELL MIELKE, MANNHEIM (DE); JOHN-PHILIP LAWO, SCHWANEWEDE (DE); BILLIE DURN, FUQUAY-VARINA, NC (US); MICHAEL TORTORICI, BERWYN, PA (US); OTHMAR ZENKER, WEIMAR (DE); IVO VAN SCHAIK, AMSTELVEEN (NL)

(73) Assignee: CSL BEHRING AG, BERN (CH)

(21) Appl. No.: 16/606,605

(22) PCT Filed: Apr. 20, 2018
(86) PCT No.: PCT/EP2018/060158

§ 371 Date: Oct. 18, 2019

Related U.S. Application Data
(60) Provisional application No. 62/488,219, filed on Apr. 21, 2017.
(60) Provisional application No. 62/571,812, filed on Oct. 13, 2017.

(30) Foreign Application Priority Data
Jun. 21, 2017 (EP) 17177134.8

(51) Int. Cl. C07K 16/06; A61P 25/00

(52) U.S. Cl. CPC C07K 16/06; A61K 45/06; C07K 2317/21; A61P 25/00

(57) ABSTRACT

The present invention relates to immunoglobulin products for use in the treatment of chronic inflammatory demyelinating polyneuropathy using immunoglobulin products. In particular, the present invention provides efficacious dosing regimens.

### FIELD OF THE INVENTION

The present inventions relates to immunoglobulin products for use in the treatment of chronic inflammatory demyelinating polyneuropathy. In particular, the present invention provides efficacious dosing regimens.

### BACKGROUND

Chronic inflammatory demyelinating polyneuropathy (CIDP) is an autoimmune disease that targets myelin sheaths, specifically in the peripheral nerves, and causes progressive weakness and sensory loss. Swelling of nerve roots is also a characteristic of the disease. Although it can occur at any age and in both genders, CIDP is more common in young adults, and it is more common in men than women.

CIDP causes peripheral neuropathy which is manifest by sensory loss, weakness, or pain, alone or in combination, in the arms, legs, or other parts of the body. It can cause a symmetric or multifocal neuropathy and affect the proximal or distal muscles. CIDP may be associated with certain other diseases. For example, it has been found that CIDP is diagnosed in one third of human immunodeficiency virus (HIV)-seropositive patients referred for peripheral nerve diseases. CIDP also occurs in subjects afflicted with lupus, paraproteinemia, lymphoma or diabetes. The course of CIDP may vary widely among individuals. Some patients may have a bout of CIDP followed by spontaneous recovery, while other patients may have many bouts with only partial recovery in between relapses.

CIDP is diagnosed based on the clinical presentation, evidence for demyelination on electrodiagnostic studies or pathological studies of biopsied nerves, and elimination of other known causes of neuropathy such as genetic defects, osteosclerotic myeloma or IgM monoclonal gammopathy.

Untreated, CIDP is characterized by accumulating disability that requires physical and occupational therapy, orthotic devices and long-term treatment. Early intervention can prevent permanent damage and disability. Current methods of treatment for CIDP include administration of corticosteroids, such as prednisone, which may be prescribed alone or in combination with immunosuppressant drugs. Immunosuppressant drugs may also be given in the absence of a steroid. Individually adjusted intravenous immunoglobulin (IVIG) therapy is also effective and is currently being used for treating CIDP. However, such current IVIG treatment requires laborious adjustment of the dosing regimen for each individual patient.

Hence, there is a need in the art for a standardized and efficacious immunoglobulin treatment of CIDP.

### SUMMARY OF THE INVENTION

The present invention is based on the unexpected finding that low fixed doses of immunoglobulin show therapeutic efficacy in the treatment of chronic inflammatory demyelinating polyneuropathy.

The present invention provides an immunoglobulin product for use in the treatment of chronic inflammatory demyelinating polyneuropathy, wherein the immunoglobulin product is to be administered at a fixed dose selected from the range of 0.1-0.4 g/kg patient weight at an interval of every 5-10 days. In a preferred embodiment, the immunoglobulin product for use in the treatment of chronic inflammatory demyelinating polyneuropathy is to be administered at a fixed dose selected from the range of 0.15-0.25 g/kg patient weight once every week. In a further preferred embodiment, the immunoglobulin product for use in the treatment of chronic inflammatory demyelinating polyneuropathy is to be administered at a fixed dose of 0.2 g/kg patient weight once every week.

In a preferred embodiment of the invention, the immunoglobulin product is administered subcutaneously. In a further preferred embodiment, patients self-administer the immunoglobulin product. Further, one dose of the immunoglobulin product may be administered all at once. Alternatively, it may be divided into several portions and be administered at various time points during one dosing interval. Accordingly, the product is convenient for patients to use and, therefore, is beneficial for patient compliance.

The provided treatment may be carried out over various time periods including weeks, months and years. It is efficacious and well tolerated. The provided treatment shows an advantageous side effect profile and, in particular, a low number of local reactions at the site of injection.

### BRIEF DESCRIPTION OF THE FIGURES

FIG. 1: Study design.

FIG. 2: Flowchart of subject disposition; CIDP=chronic inflammatory demyelinating polyneuropathy; IVIG=intravenous immunoglobulin; PP-PSDS=Per-Protocol Pre-randomization Safety Data Set; PPS=Per Protocol Set; PSDS=Pre-randomization Safety Data Set; RSDS=Rescue Medication Safety Data Set; SDS=Safety Data Set. a Exclusion of subjects from the PSDS and PPS.

FIG. 3: CIDP relapse.

FIG. 4: Kaplan-Meier Plot Time to CIDP Relapse.

FIG. 5: Summary of adverse events.

### DETAILED DESCRIPTION

### Chronic Inflammatory Demyelinating Polyneuropathy (CIDP)

CIDP is an acquired polyneuropathy within the peripheral nerve system with an assumed autoimmune-mediated pathogenesis. CIDP is characterized by symmetrical weakness in both proximal and distal muscles that worsens progressively. The condition is usually, but not always, associated with impaired sensation, absent or diminished tendon reflexes, an elevated cerebrospinal fluid protein level, and changes in electrophysiology parameters. Nerve biopsy specimens are characterized by signs of demyelination. The clinical course can be relapsing or chronic and progressive (see, e.g., Mathey E K, et al. J Neurol Neurosurg Psychiatry 2015; 86:973-985; Köller H, et al. N Engl J Med. 2005; 352(13): 1343-1356), the former being much more common in young adults. CIDP is a rare disease with an estimated prevalence of about 1.6 to 8.9 per 100,000 adults and about 0.5 per 100,000 children. CIDP may be diagnosed as described by the Joint Task Force of the EFNS and the PNS (Journal of the Peripheral Nervous System 15:1-9 (2010)).

The following conditions are identical or considered essentially identical to CIDP and are thus encompassed by the claims: “chronic relapsing polyneuropathy”, “chronic idiopathic demyelinating polyneuropathy”, “chronic inflammatory demyelinating polyradiculoneuropathy”, and “chronic acquired demyelinating polyneuropathy” (“CADP”).

### Immunoglobulin Products

The term “immunoglobulin product” is intended to mean any polyclonal antibody fraction. In this regard, the term “antibody” may be interchangeably used with the term “immunoglobulin”. The immunoglobulin product may be derived from mammalian, preferably human, plasma. In certain embodiments, the plasma of multiple (generally 1000 or more) healthy donors is pooled and optionally further processed. The term “healthy individual” means an individual who meets the current (at the time of donation) standard eligibility criteria for donating blood, bearing in mind that such eligibility criteria are subject to continuous improvement and change. In some embodiments, the immunoglobulin fraction is enriched from the pooled plasma. Preferably, the immunoglobulin is purified from the pooled plasma. More preferably, the immunoglobulin is purified and concentrated. In various embodiments, purified and concentrated immunoglobulin G (IgG) is used.

In certain embodiments, the immunoglobulin product may contain traces of immunoglobulins of different Ig classes such as IgA or IgM. In one embodiment, the IgA concentration is 50 μg or less per 100 mg immunoglobulin. In a preferred embodiment, the IgA concentration is 25 μg or less per 100 mg immunoglobulin. Low IgA is desirable in order to avoid adverse events in patients with IgA deficiency. In one embodiment, the IgM concentration is 10 μg or less per 100 mg immunoglobulin. In a preferred embodiment, the IgM concentration is 5 μg or less per 100 mg immunoglobulin. In various embodiments, the immunoglobulin product exhibits a purity of the protein fraction of >90% IgG, more preferably >95% IgG, even more preferably >98% IgG. In various embodiments, the immunoglobulin product exhibits an immunoglobulin monomer and dimer content of >90%, more preferably >95%, even more preferably >98%. The provided product preferably exhibits a natural IgG subclass distribution. In one embodiment, the immunoglobulin subclass distribution in the immunoglobulin product is 62-74% IgG1, 22-34% IgG2, 2-5% IgG3 and 1-3% IgG4. The immunoglobulin product may contain additional ingredients such as stabilizers, for example amino acids such as proline or glycine, or sucrose, maltose, sorbitol, albumin nicotinamide, PEG, polysorbate 80, or others. Preferred stabilizers are amino acids, in particular proline. In various embodiments, the immunoglobulin product contains 10-30% (w/v) immunoglobulin. In certain embodiments, the immunoglobulin product is provided as a solution containing at least 10% (w/v) immunoglobulin, more preferably at least 15% (w/v) immunoglobulin, most preferably about 20% (w/v) immunoglobulin. The immunoglobulin product may also contain about 30% (w/v) immunoglobulin. The immunoglobulin product is virus-safe for enveloped viruses (e.g., HIV, HBV and HCV) and non-enveloped viruses (e.g., HAV and parovirus B19).

The immunoglobulin product may be provided as a liquid product or a lyophilized product. In a preferred embodiment, the immunoglobulin product is provided as a liquid product. Such liquid products are ready-for-use, i.e., it is not necessary to reconstitute the product prior to administration. Liquid products are convenient to use, as no reconstitution is required. Therefore, liquid products are particularly suitable for self-administration by patients.

The provided immunoglobulin products are storage-stable over extended time periods. In one embodiment, the immunoglobulin product is storage-stable in liquid form for at least 12 months when stored at a maximum temperature of 25° C. In a preferred embodiment, the immunoglobulin product is storage-stable in liquid form for at least 24 months when stored at a maximum temperature of 25° C. In a further preferred embodiment, the immunoglobulin product is storage-stable in liquid form for at least 30 months when stored at a maximum temperature of 25° C. The term “storage-stability” as used herein refers to the maintenance of one or more features of the immunoglobulin product over the storage period. For example, storage-stability is indicated by the absence of immunoglobulin aggregation. In one embodiment, the immunoglobulin monomer and dimer content of the immunoglobulin product remains above 95% during storage for at least 12 months when stored at a maximum temperature of 25° C. In a further embodiment, the immunoglobulin monomer and dimer content of the immunoglobulin product remains above 95% during storage for at least 24 months when stored at a maximum temperature of 25° C. In one embodiment, the immunoglobulin monomer and dimer content of the immunoglobulin product remains above 98% during storage for at least 12 months when stored at a maximum temperature of 25° C. In a further embodiment, the immunoglobulin monomer and dimer content of the immunoglobulin product remains above 98% during storage for at least 24 months when stored at a maximum temperature of 25° C.

A preferred immunoglobulin product is a product for subcutaneous administration (SCIG). The term “subcutaneous immunoglobulin G”, abbreviated SCIG, means a therapeutic preparation of pooled immunoglobulin G formulated for subcutaneous administration. SCIG also denotes a product as well as a preferred route of administration (subcutaneous administration). In certain embodiments, the SCIG is VIVAGLOBIN® or HIZENTRA® (both manufactured and sold by CSL Behring).

The immunoglobulin product may also be a product for intravenous administration (IVIG). IVIG denotes a product, as well as the preferred route of administration (intravenous administration). In certain embodiments, the IVIG is PRIVIGEN® or SANDOGLOBULIN®/CARIMUNE® (both manufactured and sold by CSL Behring).

### Dosing Schemes

The present invention provides a fixed weight-based dose of an immunoglobulin product that is efficacious in the treatment of chronic inflammatory demyelinating polyneuropathy. The term “fixed dose” as used herein refers to a particular weight-based dose that can be administered to all patients. By using such a fixed dose, no individual dose adjustment is required.

In particular, the present invention provides an immunoglobulin product for use in the treatment of chronic inflammatory demyelinating polyneuropathy, wherein the immunoglobulin product is to be administered at a fixed dose selected from the range of 0.1-0.4 g/kg patient weight, from the range of 0.1-0.3 g/kg patient weight, from the range of 0.15-0.25 g/kg patient weight, from the range of 0.18-0.22 g/kg patient weight or a fixed dose of 0.2 g/kg patient weight per 5-10 days, per 6-8 days or per week.

Provided herein is an immunoglobulin product for use in the treatment of chronic inflammatory demyelinating polyneuropathy, wherein the immunoglobulin product is to be administered at a fixed dose selected from the range of 0.1-0.4 g/kg patient weight per 5-10 days. Further provided is an immunoglobulin product for use in the treatment of chronic inflammatory demyelinating polyneuropathy, wherein the immunoglobulin product is to be administered at a fixed dose selected from the range of 0.1-0.4 g/kg patient weight per 6-8 days. Further provided is an immunoglobulin product for use in the treatment of chronic inflammatory demyelinating polyneuropathy, wherein the immunoglobulin product is to be administered at a fixed dose selected from the range of 0.1-0.4 g/kg patient weight per week.

Provided herein is an immunoglobulin product for use in the treatment of chronic inflammatory demyelinating polyneuropathy, wherein the immunoglobulin product is to be administered at a fixed dose selected from the range of 0.1-0.3 g/kg patient weight per 5-10 days. Further provided is an immunoglobulin product for use in the treatment of chronic inflammatory demyelinating polyneuropathy, wherein the immunoglobulin product is to be administered at a fixed dose selected from the range of 0.1-0.3 g/kg patient weight per 6-8 days. Further provided is an immunoglobulin product for use in the treatment of chronic inflammatory demyelinating polyneuropathy, wherein the immunoglobulin product is to be administered at a fixed dose selected from the range of 0.1-0.3 g/kg patient weight per week.

Provided herein is an immunoglobulin product for use in the treatment of chronic inflammatory demyelinating polyneuropathy, wherein the immunoglobulin product is to be administered at a fixed dose selected from the range of 0.15-0.25 g/kg patient weight per 5-10 days. Further provided is an immunoglobulin product for use in the treatment of chronic inflammatory demyelinating polyneuropathy, wherein the immunoglobulin product is to be administered at a fixed dose selected from the range of 0.15-0.25 g/kg patient weight per 6-8 days. Further provided is an immunoglobulin product for use in the treatment of chronic inflammatory demyelinating polyneuropathy, wherein the immunoglobulin product is to be administered at a fixed dose selected from the range of 0.15-0.25 g/kg patient weight per week.

Provided herein is an immunoglobulin product for use in the treatment of chronic inflammatory demyelinating polyneuropathy, wherein the immunoglobulin product is to be administered at a fixed dose selected from the range of 0.18-0.22 g/kg patient weight per 5-10 days. Further provided is an immunoglobulin product for use in the treatment of chronic inflammatory demyelinating polyneuropathy, wherein the immunoglobulin product is to be administered at a fixed dose selected from the range of 0.18-0.22 g/kg patient weight per 6-8 days. Further provided is an immunoglobulin product for use in the treatment of chronic inflammatory demyelinating polyneuropathy, wherein the immunoglobulin product is to be administered at a fixed dose selected from the range of 0.18-0.22 g/kg patient weight per week.

Provided herein is an immunoglobulin product for use in the treatment of chronic inflammatory demyelinating polyneuropathy, wherein the immunoglobulin product is to be administered at a fixed dose of 0.2 g/kg patient weight per 5-10 days. Further provided is an immunoglobulin product for use in the treatment of chronic inflammatory demyelinating polyneuropathy, wherein the immunoglobulin product is to be administered at a fixed dose of 0.2 g/kg patient weight per 6-8 days. Further provided is an immunoglobulin product for use in the treatment of chronic inflammatory demyelinating polyneuropathy, wherein the immunoglobulin product is to be administered at a fixed dose of 0.2 g/kg patient weight per week.

Further provided is a method for treating chronic inflammatory demyelinating polyneuropathy (CIDP), wherein the method comprises administering an immunoglobulin product to a patient in need thereof, wherein the immunoglobulin product is to be administered at a fixed dose selected from the range of 0.1-0.4 g/kg patient weight per 5-10 days. The individual dosing schemes listed herein for the immunoglobulin product for use in the treatment of chronic inflammatory demyelinating polyneuropathy equally apply to any methods for treating chronic inflammatory demyelinating polyneuropathy.

The immunoglobulin product may be administered in any suitable way. In one embodiment, the immunoglobulin product is administered intravenously. In a preferred embodiment, the immunoglobulin product is administered subcutaneously. Subcutaneous administration may be carried out by subcutaneous bolus injection or subcutaneous infusion. Subcutaneous infusion may be carried out by using an infusion pump. Subcutaneous administration of immunoglobulin products is advantageous as it results in low peak to trough ratios in patients. Accordingly, by subcutaneous administration, the administered IgG remains at a relatively stable level in the patient. Such stable levels ensure an optimal treatment effect.

In a further preferred embodiment, the patient self-administers the immunoglobulin product. Self-administration enhances patient compliance. No visit of a treatment center is required. Further, the administration can be incorporated into the patient's daily life according to the patient's convenience.

The entire fixed dose of immunoglobulin product may be administered at once, i.e., without any discontinuation of administration. The dose may also be divided into several portions and these portions may be administered individually with breaks in between. Such stepwise administration may be carried out over the course of one day or over the course of several days. In one embodiment, a fixed dose of the immunoglobulin product is divided into two or more portions and these portions are administered over the course of 1-7 days. Accordingly, the patient may individually decide whether s/he prefers to receive the entire dose at once or to receive the dose in several portions over the course of one or several days.

In one embodiment, the fixed dose of the immunoglobulin product is to be administered over the course of 1-7 days. In a further embodiment, the fixed dose of the immunoglobulin product is to be administered over the course of one day. In a further embodiment, the fixed dose of the immunoglobulin product is to be administered over the course of two days. In a further embodiment, the fixed dose of the immunoglobulin product is to be administered over the course of three days. In a further embodiment, the fixed dose of the immunoglobulin product is to be administered over the course of four days. In a further embodiment, the fixed dose of the immunoglobulin product is to be administered over the course of five days. In a further embodiment, the fixed dose of the immunoglobulin product is to be administered over the course of six days. In a further embodiment, the fixed dose of the immunoglobulin product is to be administered over the course of seven days.

In another embodiment, two or more fixed doses are combined and are administered in accordingly extended intervals. Upon such combination, the total dose per time is likewise maintained. For example, if the total weekly dose is 0.2 g/kg patient weight, 0.4 g/kg could be administered biweekly.

In one embodiment, the fixed dose of the immunoglobulin product is doubled and it is to be administered every 14 days. In a further embodiment, the fixed dose of the immunoglobulin product is tripled and it is to be administered every 21 days. In a further embodiment, the fixed dose of the immunoglobulin product is quadrupled and it is to be administered every 28 days.

In one embodiment, the immunoglobulin product is to be administered at a flexible dosing regimen. In such a flexible dosing regimen, the total dose of immunoglobulin product administered over time is kept constant. Therefore, irrespective of whether administration occurs less frequent (increased dosing intervals) or more frequent (reduced dosing intervals), the same amount of immunoglobulin product is administered over time. The total weekly dose is maintained, although the dosing interval may be longer or shorter than one week. The invention is further described by the following embodiments:
The recommended subcutaneous dose is 0.2 to 0.4 g/kg (1 mL to 2 mL/kg) body weight per week.
Initiate therapy with immunoglobulin product 1 week after the last IGIV infusion.
Provided the total weekly dose is maintained, any dosing interval from daily up to biweekly (every 2 weeks) can be used and will result in systemic serum IgG exposure that is comparable to the weekly immunoglobulin product treatment.
Biweekly: Multiply the calculated immunoglobulin product weekly dose by 2.
Frequent dosing (2 to 7 times per week): Divide the calculated weekly dose by the desired number of times per week (e.g., for 3 times per week dosing, divide weekly dose by 3).

Hence, the provided treatment gives patients great flexibility regarding the administration schedule of the drug.

The provided treatment may be carried out over extended time periods ranging from several weeks to years. In one embodiment, the treatment is carried out for at least 3 months. In a further embodiment, the treatment is carried out for at least 6 months. In another embodiment, the treatment is carried out for at least 12 months. In yet another embodiment, the treatment is carried out for at least 24 months.

The provided treatment is well tolerated. Upon subcutaneous administration of low doses of immunoglobulin, e.g., 0.2 g/kg patient weight, local reactions at the site of injection occur only at low frequency.

Patients not responding to immunoglobulin treatment may undergo an individual dose adjustment in order to experience a treatment effect.

### Treatment Effects

The provided treatment may result in various treatment effects. These effects include: INCAT score, R-ODS score, Mean grip strength, MRC sum score (8 muscle groups) and electrophysiology parameters: distal and proximal latencies, compound action potential (CMAP) amplitudes, nerve conduction velocities, and conduction block in 3 motor nerves. These effects can be achieved with any of the dose ranges provided herein. In one embodiment, the effect is achieved with a fixed dose selected from the range of 0.1-0.4 g/kg patient weight per 6-8 days. In a preferred embodiment, the effect is achieved with a fixed dose selected from the range of 0.18-0.22 g/kg patient weight per 6-8 days.

The INCAT score is a 10-point scale that covers the functionality of legs and arms, and has been successfully used to measure treatment effects in various CIDP studies. Scores for arm disability range from 0 (“No upper limb problems”) to 5 (“Inability to use either arm for any purposeful movement”), and scores for leg disability range from 0 (“Walking not affected”) to 5 (“Restricted to wheelchair, unable to stand and walk a few steps with help”). The INCAT (total) score is the sum of these 2 scores and ranges from 0 to 10. For the “adjusted” INCAT score, changes in the function of the upper limbs from 0 (normal) to 1 (minor symptoms) or from 1 to 0 are not recorded as deterioration or improvement because these changes are not considered clinically significant (Hughes R et al, Ann Neurol. 2001; 50(2): 195-201; Hughes R A et al., Lancet Neurol. 2008; 7(2): 136-144; Hughes R A, Expert Rev Neurother. 2009; 9(6): 789-795.).

The R-ODS centile score is an outcome measure that captures activity and social participation in subjects with Guillain-Barré Syndrome, CIDP, and monoclonal gammopathy of uncertain significance related polyneuropathy (MGUSP) (van Nes S I et al. Neurology. 2011; 76(4): 337-345). This Rasch analysis-based 24-item questionnaire covers a wide range of tasks of daily life ranging from easiest tasks such as “reading a newspaper/book” and “eating” to the most difficult tasks such as “running” or “standing for hours” that are each to be rated as “impossible to perform”, “performed with difficulties”, or “easily performed”.

The Mean Grip Strength may be measured by Martin Vigorimeter. The hand-held Vigorimeter from Martin (Tuttlingen, Germany) is a device that measures the strength of small muscles in the hand; i.e., grip strength. Subjects squeeze a rubber bulb lying between the palm of the hand and the thumb and index fingers. The pressure is recorded via a rubber tube on a nanometer and expressed in kilopascal. At each assessment, the subjects squeeze 3 times with each hand. The mean grip strength of each hand is determined.

An adapted version of the MRC sum score (Leger J M et al, Brain. 2001; 124(Pt 1): 145-153) may be used. MRC sum score grades could range from 0 (“No visible contraction”) to 5 (“Normal”). The following 8 bilateral muscle pairs may be assessed, and individual muscle scores and the sum score are documented: shoulder abduction, elbow flexion, wrist extension, index finger abduction, hip flexion, knee extension, foot dorsiflexion, great toe dorsiflexion.

Electrophysiology parameters may be assessed. Three motor nerves (2 in the arm, 1 in the leg) are measured: median, ulnar, and peroneal. The stimulation points is as follows: ulnar nerve: wrist, above elbow; median nerve: wrist, elbow; peroneal nerve: ankle, below fibular head, lateral popliteal fossa.

In one embodiment, the provided treatment results in an improvement of one or more of INCAT score, R-ODS score, Mean grip strength, MRC sum score and electrophysiology parameters by at least 10% over placebo treatment. In a further embodiment, the provided treatment results in an improvement of one or more of INCAT score, R-ODS score, Mean grip strength, MRC sum score and electrophysiology parameters by at least 20% over placebo treatment. In a further embodiment, the provided treatment results in an improvement of one or more of INCAT score, R-ODS score, Mean grip strength, MRC sum score and electrophysiology parameters by at least 30% over placebo treatment. In a further embodiment, the provided treatment results in an improvement of one or more of INCAT score, R-ODS score, Mean grip strength, MRC sum score and electrophysiology parameters by at least 40% over placebo treatment. In a further embodiment, the provided treatment results in an improvement of one or more of INCAT score, R-ODS score, Mean grip strength, MRC sum score and electrophysiology parameters by at least 50% over placebo treatment. In a further embodiment, the provided treatment results in an improvement of one or more of INCAT score, R-ODS score, Mean grip strength, MRC sum score and electrophysiology parameters by at least 60% over placebo treatment. In a further embodiment, the provided treatment results in an improvement of one or more of INCAT score, R-ODS score, Mean grip strength, MRC sum score and electrophysiology parameters by at least 70% over placebo treatment. In a further embodiment, the provided treatment results in an improvement of one or more of INCAT score, R-ODS score, Mean grip strength, MRC sum score and electrophysiology parameters by at least 80% over placebo treatment. In a further embodiment, the provided treatment results in an improvement of one or more of INCAT score, R-ODS score, Mean grip strength, MRC sum score and electrophysiology parameters by at least 90% over placebo treatment. In a further embodiment, the provided treatment results in an improvement of one or more of INCAT score, R-ODS score, Mean grip strength, MRC sum score and electrophysiology parameters by at least 100% over placebo treatment.

The provided treatment results in a reduction of the CIDP relapse rate in CIDP patients. In one embodiment, the provided treatment with a fixed dose selected from the range of 0.1-0.4 g/kg per 6-8 days results in a reduction in the relapse rate of more than 20%, preferably more than 30%, more preferably more than 40%, or even more than 50% when compared to placebo. In a further embodiment, the provided treatment with a fixed dose selected from the range of 0.18-0.22 g/kg per 6-8 days results in a reduction in the relapse rate of more than 20%, preferably more than 30%, more preferably more than 40% when compared to placebo.

In one embodiment, the provided treatment with a fixed dose selected from the range of 0.18-0.22 g/kg per 6-8 days results in a reduction in the relapse rate of more than 20% when compared to placebo. In one embodiment, the provided treatment with a fixed dose selected from the range of 0.18-0.22 g/kg per 6-8 days results in a reduction in the relapse rate of more than 30% when compared to placebo. In one embodiment, the provided treatment with a fixed dose selected from the range of 0.18-0.22 g/kg per 6-8 days results in a reduction in the relapse rate of more than 40% when compared to placebo. In one embodiment, the provided treatment with a fixed dose of 0.2 g/kg weekly results in a reduction in the relapse rate of more than 20% when compared to placebo. In one embodiment, the provided treatment with a fixed dose of 0.2 g/kg weekly results in a reduction in the relapse rate of more than 30% when compared to placebo. In one embodiment, the provided treatment with a fixed dose of 0.2 g/kg weekly results in a reduction in the relapse rate of more than 40% when compared to placebo.

The provided treatment results in an increase of patients who do not experience a CIDP relapse. In one embodiment, the provided treatment with a fixed dose selected from the range of 0.1-0.4 g/kg per 6-8 days results in an increase of patients who do not experience a CIDP relapse of more than 30%, preferably more than 40%, more preferably more than 60%, or even more than 80% when compared to placebo. In one embodiment, the provided treatment with a fixed dose selected from the range of 0.18-0.22 g/kg per 6-8 days results in an increase of patients who do not experience a CIDP relapse of more than 30%, preferably more than 40%, more preferably more than 50%, or even more than 60% when compared to placebo.

The provided treatment results in a reduction of the probability of CIDP relapse in CIDP patients over extended time periods in comparison to placebo treated patients. In one embodiment, the provided treatment with a fixed dose selected from the range of 0.1-0.4 g/kg per 6-8 days results in a reduction of the probability of relapse after 5 weeks of treatment by more than 10%, preferably more than 15%, more preferably more than 20%, or even more than 25% when compared to placebo. In one embodiment, the provided treatment with a fixed dose selected from the range of 0.18-0.22 g/kg per 6-8 days results in a reduction of the probability of relapse after 5 weeks of treatment by more than 10%, preferably more than 12%, more preferably more than 13%, or even more than 15% when compared to placebo. In one embodiment, the provided treatment with a fixed dose selected from the range of 0.1-0.4 g/kg per 6-8 days results in a reduction of the probability of relapse after 14 weeks of treatment by more than 10%, preferably more than 20%, more preferably more than 30%, or even more than 40% when compared to placebo. In one embodiment, the provided treatment with a fixed dose selected from the range of 0.18-0.22 g/kg per 6-8 days results in a reduction of the probability of relapse after 14 weeks of treatment by more than 10%, preferably more than 15%, more preferably more than 18%, or even more than 20% when compared to placebo. In one embodiment, the provided treatment with a fixed dose selected from the range of 0.1-0.4 g/kg per 6-8 days results in a reduction of the probability of relapse after 24 weeks of treatment by more than 10%, preferably more than 20%, more preferably more than 30%, or even more than 35% when compared to placebo. In one embodiment, the provided treatment with a fixed dose selected from the range of 0.18-0.22 g/kg per 6-8 days results in a reduction of the probability of relapse after 24 weeks of treatment by more than 15%, preferably more than 20%, more preferably more than 23%, or even more than 24% when compared to placebo.

### EXAMPLES

### Patients

Patients were eligible if they were at least 18 years old and had been diagnosed with definite or probable CIDP according to the European Federation of Neurological Societies/Peripheral Nerve Society (EFNS/PNS) criteria 2010 (Van den Bergh P Y K, et al; Eur J Neurol 2010; 17:356-63) and if they responded to IVIg treatment as assessed by the treating physician within 8 weeks before enrollment. During the conduct of this trial, the protocol was amended five times. The amendments did not impact the randomized treatment period apart from an increase of sample size.

### Trial Design

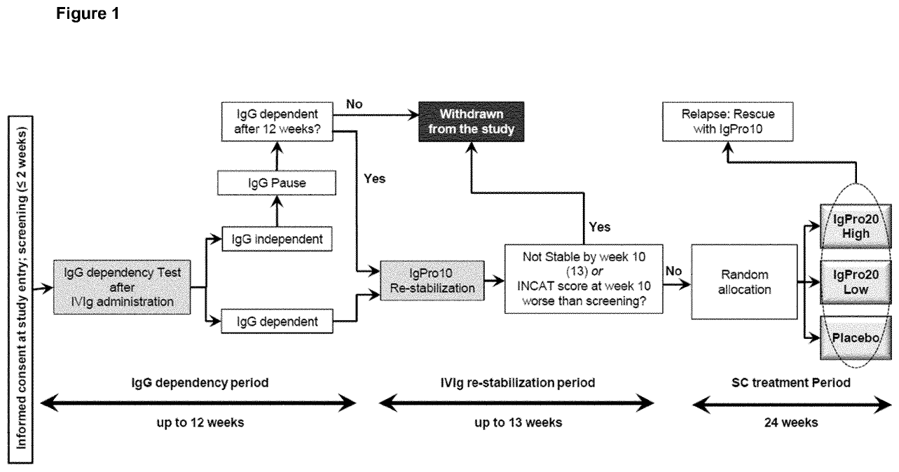

The conducted study was an international multicenter, double-blind, randomized placebo-controlled phase III study. After screening, all eligible patients progressed through an IgG dependency test period. Only patients who were determined to be IgG dependent were enrolled into the IVIG re-stabilization period. This period was performed with IgPro10 (Privigen®, CSL Behring, Bern, Switzerland) using the EFNS/PNS guideline recommended dose (Van den Bergh P Y K, et al; Eur J Neurol 2010; 17:356-63). Only patients who improved to at least the INCAT total score recorded at screening visit and who maintained a stable INCAT total score during the last three weeks of the re-stabilitzation period were eligible for randomization (FIG. 1).

Patients were randomly assigned in a 1:1:1 ratio using block randomization with a block size of six, stratified for region (Japan/non-Japan) by means of an interactive voice/web-response system to receive high or low dose IgPro20 (Hizentra®, CSL Behring, Bern, Switzerland) or placebo. A completion visit was performed for all patients following SC completion or withdrawal for any reason during the SC treatment period.

### Treatment and Blinding

IgPro20 or placebo were self-administered or administered by a care-giver at home, after appropriate site training. During the SC treatment period, the total dose/volume for all groups was based on body weight. One group received IgPro20 at 0.4 g/kg, one group received IgPro20 at 0.2 g/kg plus placebo to match volume in all three groups, and one group received only placebo. Weekly SC infusions were performed during 1 or 2 consecutive days in 2 sessions using infusion pumps. All patients and study personnel were blinded and unaware of treatment assignment. Standard measures were taken for placebo (2% human albumin solution) and IgPro20 to ensure adequate blinding. A “two-physician” approach was implemented to reduce the chance of potential study unblinding and minimize bias. The “treating” physician was the primary contact for the patient and was responsible for all patient-related questions, adverse event (AE) evaluation, and for all other study-related tasks. A second “evaluating” physician was responsible for assessment of efficacy variables. The evaluating physician did not have access to any data collected by the treating physician.

### Outcome Measures and Data Collection

The primary outcome was defined as the percentage of patients who experienced a CIDP relapse during SC treatment or who were withdrawn from the study during SC treatment for any reason.

CIDP relapse was defined as a deterioration from baseline (ie, increase) by at least 1 point in the total adjusted INCAT score (Hughes R A, et al; The Lancet Neurol 2008; 7:136-44). Baseline scores were defined as the scores assessed at the end of the IVIg re-stabilization period. Secondary outcomes for the SC treatment period were time to the primary endpoint, between-group differences of the median changes from baseline to completion visits in INCAT score, mean grip strength for both hands separately as assessed using the handheld Martin Vigorimeter (Vanhoutte E K, et al; Eur J Neurol 2013; 20:748-55.), Medical Research Council (MRC) sum score (Kleyweg R P, et al; Muscle & Nerve 1991; 14:1103-9) and inflammatory neuropathy-Rasch-built Overall Disability Scale (I-RODS) (Van Nes S I, et al; Neurology 2011; 76:337-45). Primary and secondary outcome measures were assessed at screening; during the IgG dependency test period, before IVIg infusions during the IVIg re-stabilization period; at baseline; at all visits during the SC treatment period including the completion visit; and at any unscheduled visit.

Quality of life was assessed using the EuroQoL 5-Dimension Questionnaire (EQ-5D), Treatment Satisfaction Questionnaire for Medication (TSQM) and Work Productivity and Activity Impairment Questionnaire for General Health (WPAI-GH) (van Schaik I N, et al; Trials 2016; 17:345).

To assess safety and tolerability of IgPro20 versus placebo, adverse events (AEs) per infusion and the number and percentage of patients with AEs were determined. All outcomes for the two pre-randomization periods, rescue treatment and other exploratory outcomes will be reported separately.

### Statistical Analysis

Sample size calculation was based on the null hypothesis that the percentage of relapsed or withdrawn patients during SC treatment was non-increasing from placebo to low dose to high dose arm, with at least one of the examined SCIg dose arms have a strictly lower percentage than the placebo arm. It was assumed that the percentages of patients who reached the primary endpoint was 35% for the high dose, 52% for the low dose, and 65% for placebo (van Schaik I N, et al; Trials 2016; 17:345). These numbers were based on data of the ICE study extension period (Hughes R A, et al; The Lancet Neurol 2008; 7:136-44). Using the exact Cochran-Armitage trend test with equally spaced scores and a one-sided significance level of 0.025, a sample size of 58 was needed in each treatment arm to achieve a power of 90% in an intention-to-treat analysis based on the above assumptions. Accounting for patients who would not pass the IgG dependency test and IVIg re-stabilization period, it was expected that up to 350 patients would need to be screened to ensure that 174 patients were randomized.

The exact Cochran-Armitage trend test was used for the primary outcome to test for a trend over the three trial arms at a one-sided type-I error of 0.025. If the hypothesized superiority was demonstrated, one-sided Fisher's exact tests were used for the subsequent pairwise comparisons: placebo vs. low dose, placebo vs. high dose, and low dose vs. high dose. The proportions and corresponding two-sided 95% Wilson-Score confidence intervals were calculated for each treatment group. Point estimates for the difference in proportions and the corresponding exact two-sided 95% confidence intervals were calculated for all pair-wise treatment comparisons. Three pre-specified sensitivity analyses with modified primary endpoint definitions investigated the potential bias for any reason other than CIDP relapse (van Schaik I N, et al; Trials 2016; 17:345). Complementary to the primary analysis and the sensitivity analyses, two time-to-event analyses were performed and Kaplan-Meier estimates were derived. In the first patients who withdrew for other reasons were considered to have reached the endpoint, in the second withdrawals for other reasons contributed to a censored outcome.

Secondary endpoints were presented as median changes from baseline and compared between the three groups using the asymptotic Jonckheere-Terpstra test (Jonckheere A R; Biometrika 1954; 41:133-45). Pairwise comparisons based on median changes from baseline were done using one-sided Wilcoxon rank sum tests. Statistical testing was adjusted for multiple testing for the primary endpoint only. All other comparisons are therefore considered exploratory.

The primary outcome, including all sensitivity analyses, was assessed in the intention-to-treat set (ITTS) and per protocol set (PPS) (van Schaik I N, et al; Trials 2016; 17:345). Safety was assessed in the safety data set including all randomized patients who received at least one dose of IgPro20/placebo.

### Results

### Patients

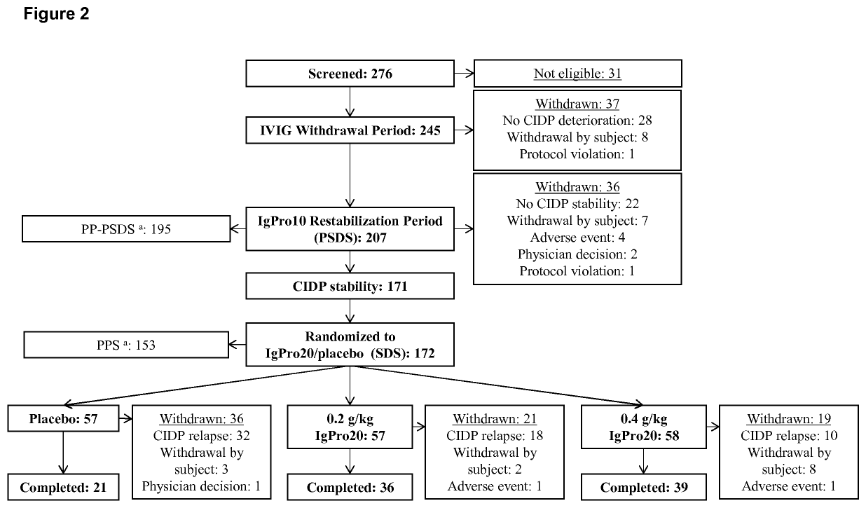

Patients were enrolled in 69 centers worldwide from March 2012 until November 2015, with last patient visit in September 2016. A total of 276 unique patients were screened. In total, 172 patients randomized (FIG. 2). All these 172 patients received their allocated treatment and 99.7% of planned volumes were actually administered. Patients tolerated volumes up to 50 ml per injection site with two to eight infusion sites running in parallel and up to 50 ml/h/site infusion rate, maximum total infusion volume was 140 ml. Infusion time was approximately one hour. No patients were lost to follow-up. Table 1 shows the baseline characteristics of all randomized patients.

TABLE 1
Baseline characteristics

| | | | Low dose | | High dose | |
|---|---|---|---|---|---|---|
| | Placebo | | IgPro20 | | IgPro20 | |
| | n = 57 | | n = 57 | | n = 58 | |
| Male | 37 | (64.9) | 42 | (73.7) | 31 | (53.4) |
| Age (years) | 57.6 | (28.6-77.0) | 58.9 | (25.8-77.6) | 55.2 | (24.7-82.7) |
| Body weight (kg) | 85.8 | (17.4) | 83.3 | (16.6) | 79.4 | (21.2) |
| BMI (kg/m²) | 28.4 | (4.6) | 27.6 | (4.9) | 26.3 | (5.5) |
| Duration of disease (years) | 2.7 (Q1, Q3 | | 2.8 (Q1, Q3 | | 3.3 (Q1, Q3 | |
| | 1.1, 4.7) | | 1.4, 5.0) | | 1.3, 8.6) | |
| EFNS/PNS CIDP criteria | | | | | | |
| Definite | 53 | (93.0) | 51 | (89.5) | 53 | (91.4) |
| Probable | 4 | (7.0) | 6 | (10.5) | 5 | (8.6) |
| Patients with ≥4 IVIg treatments | 51 | (89.5) | 52 | (91.2) | 54 | (93.1) |
| in 9 months before enrolment | | | | | | |
| IVIg dose during 3 months prior | 2.4 | (1.5) | 2.4 | (1.5) | 2.7 | (1.5) |
| to screening (g/kg) | | | | | | |
| INCAT disability scale (possible | 2 (Q1, Q3 | | 2 (Q1, Q3 | | 2 (Q1, Q3 | |
| range 0-10) | 1, 3) | | 1, 3) | | 1, 3) | |
| I-RODS (possible range 0-100)^{#} | 68 (Q1, Q3 | | 63 (Q1, Q3 | | 69 (Q1, Q3 | |
| | 51-83) | | 51, 73) | | 54, 80) | |
| Grip strength Dominant hand | | | | | | |

| | | | |
|---|---|---|---|
| (kPa; possible range 0-160) | 68.0 (Q1, Q3 | 67.0 (Q1, Q3 | 68.4 (Q1, Q3 |
| | 49.3, 83.7) | 56.7, 86.2) | 46.0, 93.3) |
| MRC sum score (possible range | 76 (Q1, Q3 | 75 (Q1, Q3 | 76 (Q1, Q3 |
| 0-80) | 72, 78) | 70, 78) | 70, 79) |

Data are number (%), mean (SD), or median (range) unless otherwise stated. Larger INCAT disability scale values indicate greater limitation. Larger MRC sum score indicates greater strength. The I-RODS scores range from 0 indicating most severe activity and social participation limitations to 100 if a patient is fully able.
IQR = interquartile range. Q1 = 1st quartile, Q3 = 3rd quartile.
^{#}total n = 152: missing data in 11 placebo, 6 low dose and 3 high dose patients.

### Efficacy

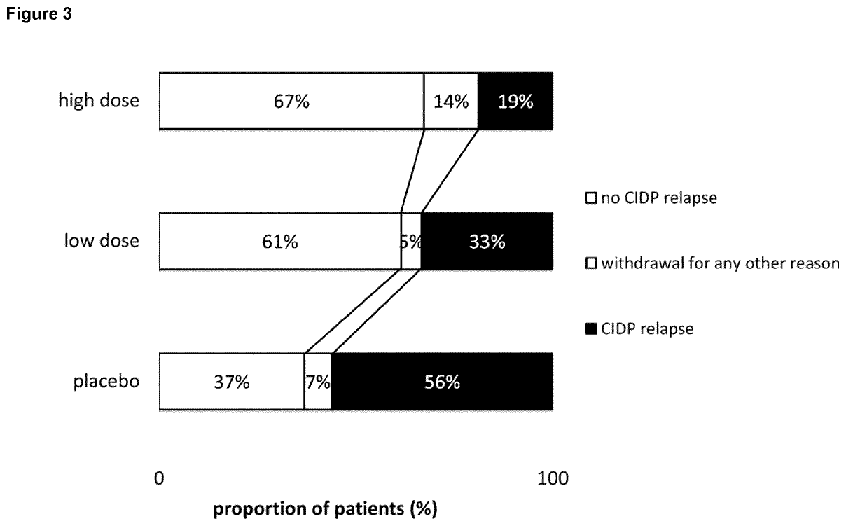

Seventy-seven patients had a CIDP relapse or were withdrawn from the study: 36 (63%) in the placebo group, 22 (39%) in the low-dose group and 19 (33%) in the high-dose group (FIG. 3, Table 2). The absolute risk reduction (ARR) for reaching the primary endpoint was 24.6% (95% CI 6.2, 40.7) in the low dose group and 30.4% (12.2, 46.0) in the high dose group as compared with placebo. Comparing low dose with high dose, the ARR was 5.8 (−11.4, 22.6). Both SCIG doses were superior to placebo (p=0.007 and p<0.001). The sensitivity analyses showed that the patients, who withdrew for reasons other than relapse, did not influence the primary endpoint outcome (Table 2).

TABLE 2
Primary outcome

| | %, 95% CI (number of patients) | | | Exact | Fisher's exact test (p) | | |
|---|---|---|---|---|---|---|---|
| | | | | Cochran- | | | High- |
| | | Low- | High- | Armitage | Low- | High- | dose |
| | | dose | dose | test (p) | dose | dose | vs |
| | Placebo | SCIG | SCIG | Overall | vs | vs | low- |
| | n = 57 | n = 57 | n = 58 | test | placebo | placebo | dose |
| Primary | 63, 50.2-74.5 | 39, 27.1-51.6 | 33, 22.1-45.6 | <0.001 | 0.007 | <0.001 | 0.323 |
| outcome | (36) | (22) | (19) | | | | |
| ITT | | | | | | | |
| | n = 52 | n = 54 | n = 47 | | | | |
| PP | 64, 50.0-75.2 | 39, 27.0-52.2 | 26, 15.3-39.5 | <0.001 | 0.010 | <0.001 | 0.112 |
| | (33) | (21) | (12) | | | | |
| | N = 57 | N = 57 | N = 58 | | | | |
| Sensitivity | 56, 43.3-68.2 | 33, 22.5-46.3 | 19, 10.9-30.9 | <0.001 | 0.012 | <0.001 | 0.061 |
| analysis A | (32) | (19) | (11) | | | | |
| “CIDP | | | | | | | |
| relapse | | | | | | | |
| analysis” | | | | | | | |
| Sensitivity | 60, 46.7-71.4 | 33, 22.5-46.3 | 24, 15.0-36.5 | <0.001 | 0.004 | <0.001 | 0.188 |
| analysis B | (34) | (19) | (14) | | | | |
| “mixed- | | | | | | | |
| case | | | | | | | |
| analysis” | | | | | | | |
| | n = 53 | n = 54 | n = 50 | | | | |
| Sensitivity | 60, 46.9-72.4 | 35, 23.8-48.5 | 22, 12.8-35.2 | <0.001 | 0.008 | <0.001 | 0.102 |
| analysis C | (32) | (19) | (11) | | | | |
| “complete- | | | | | | | |
| case | | | | | | | |
| analysis” | | | | | | | |
| | | | | log-rank test | | | |
| | | | | for trend | regular log-rank test | | |
| Probability | 63.2 | 39.0 | 33.7 | <0.001 | 0.007 | <0.001 | 0.483 |
| of primary | (50.9, 75.4) | (27.7, 53.1) | (22.8, 47.8) | | | | |
| outcome at | | | | | | | |
| 24 weeks^{#} | | | | | | | |
| Hazard | | | | | 0.49 | 0.38 | 0.8 |
| ratio | | | | | (0.29, 0.84) | (0.22, 0.67) | (0.43, 1.49) |
| (95% CI) | | | | | | | |
| Probability | 58.8 | 35.0 | 22.4 | <0.001 | 0.009 | <0.001 | 0.089 |
| of a | (46.1, 72.0) | (23.9, 49.3) | (12.9, 37.2) | | | | |
| relapse at | | | | | | | |
| 24 weeks^{#} | | | | | | | |
| Hazard | | | | | 0.48 | 0.25 | 0.53 |
| ratio | | | | | (0.27, 0.85) | (0.12, 0.49) | (0.25, 1.12) |
| (95% CI) | | | | | | | |

Three pre-specified sensitivity analyses with modified primary endpoint definitions investigated the potential bias for any reason other than CIDP relapse):
(A) “CIDP relapse analysis”, all patients who did not experienced a CIDP relapse were considered as non-relapsers;
(B) “mixed-case analysis”, patients who had a relapse including patients who were withdrawn because the investigator advised that the patient's safety or well-being could be compromised by further participation in the study or who received prohibited medication were compared to patients without a relapse including all patients who were withdrawn for any other reasons;
(C) “complete-case analysis”, patients with a relapse were compared with those without a relapse, excluding from analysis all patients who were withdrawn from the study. Exact Cochran-Armitage tested for a trend with superiority of at least one IgPro20 dose over placebo.
^{#}Kaplan-Meier estimates (%, (95% CI)).
All tests are one-sided with statistical significance defined at a p-value of <0.025.
Baseline scores were the last scores before randomization.
ITT = intention treat analysis;
PP = per protocol analysis;
SCIg = subcutaneous immunoglobulins.

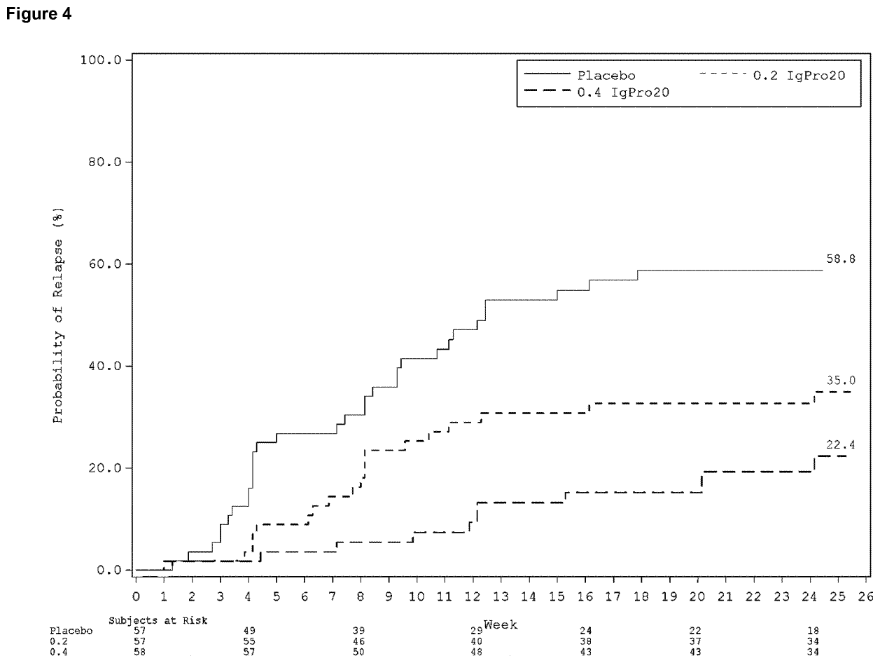

The probability to reach the primary endpoint as assessed by a time to event analysis was significantly lower in both SCIG groups than in the placebo groups (hazard ratio low-dose versus placebo: 0.49 (95% CI 0.29-0.84, p=0.007); high-dose versus placebo: 0.38 (0.22-0.67, p<0.001); FIG. 4, Table 2).

A complementary time to relapse analysis was performed censoring all withdrawals at time of withdrawal. The Kaplan-Meier estimates for CIDP relapse alone was 22% in high-dose SCIg, 35% in low-dose SCIg and 59% in placebo patients (Table 2). Both IgPro20 doses were associated with lower relapse rates as compared to placebo and the probability of a relapse was lower at all time points between SC week 3 and 25 in IgPro20-treated patients when compared with placebo-treated patients. All per protocol analyses supported the results of the ITT analysis.

The median changes from baseline in secondary outcome variables showed similar patterns as the primary outcome across the different treatment groups (Table 3). All median changes with high- and low-dose were significantly better than with placebo except for the median change with low-dose in the I-RODS scores. No significant differences were observed between the two dose groups.

TABLE 3
Secondary outcomes

| | | | | Asymptotic | Wilcoxon rank sum test (p) | | |
|---|---|---|---|---|---|---|---|
| | | | | Jonckheere- | | | High- |
| | | Low- | High- | Terpstra | Low- | High- | dose |
| | | dose | dose | test (p) | dose | dose | vs |
| | Placebo | SCIg | SCIg | Overall | vs | vs | low- |
| | n = 57 | n = 57 | n = 58 | test | placebo | placebo | dose |
| | INCAT [total score] | | | | | | |
| Value at | 3.0 (3.0, | 3.0 (2.0, | 2.0 (1.0, | <0.001 | 0.005 | <0.001 | 0.102 |
| endpoint | 4.0) | 4.0) | 3.0) | | | | |
| Change | 1.0 (0.0, | 0.0 (0.0, | 0.0 (0.0, | | | | |
| from | 2.0) | 1.0) | 0.0) | | | | |
| baseline, | | | | | | | |
| median | | | | | | | |
| (Q1, Q3)* | | | | | | | |
| | I-RODS [centile score] | | | | | | |
| Value at | 60.0 (45.0, | 61.0 (55.0, | 65.0 (52.0, | <0.001 | 0.030 | <0.001 | 0.041 |
| endpoint | 69.0) | 69.0) | 80.0) | | | | |
| Change | −3.0 (−16.0, | −2.0 (−7.0, | 0.0 (−2.0, | | | | |
| from | 0.0) | 2.0) | 3.5) | | | | |
| baseline, | | | | | | | |
| median | | | | | | | |
| (Q1, Q3)* | | | | | | | |
| | Grip strength [kPa, dominant hand] | | | | | | |
| Value at | 62.0 (36.0, | 64.0 (55.5, | 66.7 (43.3, | 0.022 | 0.004 | 0.014 | 0.198 |
| endpoint | 75.3) | 87.0) | 90.7) | | | | |
| Change | −6.6 (−21.6, | −0.6 (−8.9, | −2.7 (−6.6, | | | | |
| from | 0.3) | 7.0) | 2.0) | | | | |
| baseline, | | | | | | | |
| median | | | | | | | |
| (Q1, Q3)* | | | | | | | |
| | MRC [sum score] | | | | | | |
| Value at | 73.0 (66.0, | 74.0 (67.5, | 76.0 (68.0, | 0.003 | 0.003 | 0.002 | 0.465 |
| endpoint | 77.0) | 78.0) | 80.0) | | | | |
| Change | −2.0 (−6.0, | 0.0 (−2.0, | 0.0 (−2.0, | | | | |
| from | 0.0) | 2.0) | 1.0) | | | | |
| baseline, | | | | | | | |
| median | | | | | | | |
| (Q1, Q3)* | | | | | | | |

INCAT = Inflammatory Neuropathy Cause and Treatment;
MRC = Medical Research Council;
R-ODS = Rasch-built Overall Disability Scale;
SC = subcutaneous;
SCIg = subcutaneous immunoglobulins.
Baseline scores were the last scores before randomization.
All tests are one-sided with statistical significance defined at an unadjusted p-value of <0.025 (statistical testing was not adjusted for multiple testing for the secondary endpoint. These comparisons are therefore considered exploratory).
*at last SC post-dose observation

Health related quality of life measures generally showed better outcomes for both SCIG groups over placebo (details of patient reported outcomes not shown).

### Safety

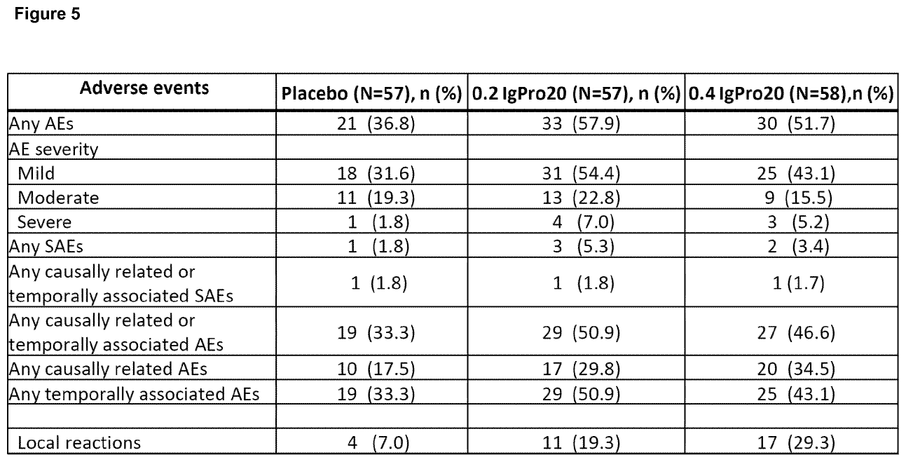

In the placebo group 21 (36.8%) patients had 52 adverse events over 1514 infusions. In the low-dose group 33 (57.9%) patients had 158 adverse events over 2007 infusions; in the high-dose group 30 (51.7%) patients had 114 adverse events over 2218 infusions. Local reactions at the infusion site occurred in 18.6% of patients and more frequently in SCIg groups (19% of low dose and 29% of high dose patients compared to 7% of placebo patients, FIG. 5). All local reactions were either mild (94.5%) or moderate (5.5%), frequency decreased during the first eight infusions, and none led to discontinuation. Eleven serious AEs were encountered in one placebo patient, three low-dose and two high-dose patients. Only one of those 11 SAEs was assessed to be causally related: in the low-dose group one patient developed an acute allergic skin reaction. This SAE led to discontinuation of treatment. No hemolysis or thrombosis occurred during the SC treatment period.

### DISCUSSION

The reported study provides evidence of the efficacy and safety of IgPro20 in preventing relapse in CIDP. In the 24 week randomized, placebo-controlled SC Treatment Period, both IgPro20 doses demonstrated superiority over placebo, with the difference being statistically significant for both IgPro20 doses.

The absolute risk reduction for CIDP relapse was 25% for the 0.2 g/kg IgPro20 dose and 30% for the 0.4 g/kg IgPro20 dose, compared with placebo. This result was achieved using a conservative endpoint including not only CIDP relapse but also subjects being withdrawn for any other reason.

When only considering CIDP relapse, the 0.2 g/kg IgPro20 dose prevented CIDP relapse in 67% of subjects and the 0.4 g/kg IgPro20 dose in 81% of subjects (FIG. 3). The absolute risk reduction compared with placebo was 23% for the 0.2 g/kg IgPro20 dose and 37% for the 0.4 g/kg IgPro20 dose. Based on the inverse of these absolute risk reductions, the number of subjects needed to treat to prevent CIDP relapse is 4 to 5 for the 0.2 g/kg IgPro20 dose and 2 to 3 for the 0.4 g/kg IgPro20 dose.

Time to CIDP relapse (FIG. 4) was evaluated, and the corresponding probabilities for CIDP relapse based on Kaplan-Meier estimates were: placebo, 58.8%; 0.2/kg bw IgPro20 35.0%; and 0.4 g/kg bw IgPro20, 22.4%. The hazard ratios (95% CI) for the lower dose and higher dose compared to placebo was 0.48 (0.27, 0.85) and 0.25 (0.12, 0.49), respectively. The difference observed between the 0.2 g/kg bw and the 0.4 g/kg bw IgPro20 groups did not reach statistical significance.

The risk of CIDP relapse was lower in each of the IgPro20 dose groups compared with placebo at all time points between Week 3 and Week 25. Based on the hazard ratios, subjects in the placebo group had a 2 times greater risk for CIDP relapse than subjects in the 0.2 g/kg IgPro20 group and a 4 times greater risk than subjects in the 0.4 g/kg IgPro20 group.

The secondary endpoints of INCAT score, R-ODS centile score, mean grip strength, and MRC sum score favored both IgPro20 doses over placebo, as did the quality of life and electrophysiology parameters.

No statistically significant differences were observed in any measures between either IgPro20 doses. Surprisingly, the dose of 0.2 g/kg IgPro20 dose is similarly efficacious as the 0.4 g/kg IgPro20 dose.

More than 88% of subjects indicated that SC infusions are easy to use.

Overall, SC treatment with IgPro20 was not only efficacious in preventing CIDP relapse but was also a preferred treatment over IVIG.

The adverse events, laboratory parameters, and vital signs observed during the study are consistent with the known safety profile of IgPro20. The most frequent AEs were local reactions, which occurred more often in IgPro20 treated subjects than in placebo treated subjects. Upon administration of the 0.2 g/kg IgPro20, the occurrence of Local Reaction AEs is reduced in comparison to administration of the 0.4 g/kg IgPro20. The frequencies of Local Reaction AEs were generally highest at the earlier infusions in the SC Treatment Period, and declined thereafter. The frequency of causally related AEs was low, most AEs were mild or moderate, and few SAEs were reported.

SC treatment was well tolerated when given in high volumes of up to 140 mL per infusion session. Subjects generally used 4 injections sites (maximum: 9 sites) and infused an average of 20 mL per site (maximum: 50 mL), with an infusion rate of 20 mL/h (maximum: 50 mL/h). The infusion time was approximately 1 hour.

Collectively the available body of evidence in this study demonstrates the efficacy and safety of IgPro20 in preventing CIDP relapse in subjects.

1-34. (canceled)

35. A method for treating chronic inflammatory demyelinating polyneuropathy (CIDP), comprising administering an immunoglobulin product to a patient in need thereof, wherein the immunoglobulin product is administered at a fixed dose of 0.1-0.4 g/kg patient weight every 5-10 days.

36. The method of claim 35, wherein the immunoglobulin product is administered every 6-8 days.

37. The method of claim 35, wherein the immunoglobulin product is administered every week.

38. The method of claim 35, wherein the immunoglobulin product is administered at a fixed dose of 0.1-0.3 g/kg patient weight.

39. The method of claim 35, wherein the immunoglobulin product is administered at a fixed dose of 0.15-0.25 g/kg patient weight.

40. The method of claim 35, wherein the immunoglobulin product is administered at a fixed dose of 0.18-0.22 g/kg patient weight.

41. The method of claim 35, wherein the immunoglobulin product is administered at a fixed dose of 0.2 g/kg patient weight.

42. The method of claim 35, wherein the immunoglobulin product is administered at a fixed dose of 0.2 g/kg patient weight per week.

43. The method of claim 35, wherein the immunoglobulin product is administered subcutaneously.

44. The method of claim 35, wherein the fixed dose of the immunoglobulin product is administered over the course of 1-7 days.

45. The method of claim 44, wherein a dose of the immunoglobulin product is administered over the course of one day.

46. The method of claim 35, wherein the treatment is carried out for at least 3 months.

47. The method of claim 35, wherein the treatment results in an improvement in one or more of INCAT score, R-ODS score, Mean grip strength, MRC sum score, and electrophysiology parameters by at least 20% compared to placebo treatment.

48. The method of claim 35, wherein the immunoglobulin product is a liquid ready-for-use product and/or the immunoglobulin product does not require reconstitution to liquid form prior to administration.

49. (canceled)

50. The method of claim 35, wherein the patient self-administers the immunoglobulin product.

51. (canceled)

52. The method of claim 35, wherein the immunoglobulin product has a concentration of 10-30% immunoglobulin.

53. The method of claim 52, wherein the immunoglobulin product has a concentration of 20% immunoglobulin.

54-56. (canceled)

57. The method of claim 35, wherein the immunoglobulin product comprises a stabilizer, and wherein the stabilizer is an amino acid, preferably wherein the stabilizer is proline.

58. (canceled)

59. The method of claim 35, wherein the immunoglobulin product is derived from human plasma or a human plasma concentrate.

60. A method for treating chronic inflammatory demyelinating polyneuropathy (CIDP), comprising administering an immunoglobulin product to a patient in need thereof, wherein the immunoglobulin product is administered in a flexible dosing regimen, wherein the total weekly dose is maintained at a dose of 0.1-0.4 g/kg patient weight.

61. The method of claim 60, wherein the total weekly dose is 0.1-0.3 g/kg patient weight.

62-64. (canceled)

65. The method of claim 60, wherein the immunoglobulin product is administered biweekly and the dose administered is the total weekly dose multiplied by 2.

66. The method of claim 60, wherein the immunoglobulin product is administered every 3 weeks and the dose administered is the total weekly dose multiplied by 3.

67. The method of claim 60, wherein the immunoglobulin product is administered twice a week and the dose administered is the total weekly dose divided by 2.

68. The method of claim 60, wherein the immunoglobulin product is administered 2-7 times per week and the total weekly dose is maintained.
